(19) United States
(12) Patent Application Publication
(10) Pub. No.: US 2019/0275257 A1
(43) Pub. Date: Sep. 12, 2019

(54) Adipose tissue transplantatioin device

(71) Applicant: Mark Jordan, Groveland, FL (US)

(72) Inventor: Mark Jordan, Groveland, FL (US)

(21) Appl. No.: 16/294,439

(22) Filed: Mar. 6, 2019

Related U.S. Application Data
(60) Provisional application No. 62/640,357, filed on Mar. 8, 2018.

(51) Int. Cl. A61M 5/315; A61B 10/02; A61K 35/35

(52) U.S. Cl. CPC A61M 5/31511; A61B 2017/00969; A61K 35/35; A61B 10/0283

(57) ABSTRACT

An adipose or fat tissue transplantation device is disclosed. The device employs a cylindrical vessel for housing a conventional piston operated syringe. A handle is pivotally attached to the cylindrical vessel with a proximal end secured to a puller positioned to engage steps attached to the piston. A distal end of the handle provides a thumb operated lever for advancing the piston a predetermined distance upon depressing the handle to move the piston. The handle includes a link arm of a length sized to move the puller upon engaging a step a predetermined distance. A spring is coupled to the link arm to maintain the distal end of the handle in a normally raised position. In a preferred embodiment, the link arm and steps are constructed and arranged to advance the piston to cause a predetermine dispensing of tissue for each handle depression.

### PRIORITY CLAIM

In accordance with 37 C.F.R. § 1.76, a claim of priority is included in an Application Data Sheet filed concurrently herewith. Accordingly, the present invention claims priority to U.S. Provisional Patent Application No. 62/640,357, entitled “ADIPOSE TISSUE TRANSPLANTATION DEVICE”, filed Mar. 8, 2018. The content of the above-referenced application are incorporated herein by reference in its entirety.

### FIELD OF THE INVENTION

The present invention relates to the field of body contouring devices and, more specifically, to a device for implantation of fat tissue.

### BACKGROUND

Cosmetic surgery used for body contouring is well known. Liposuction is a popular cosmetic surgery used in removing adipose tissue. Adipose tissue, also referred to as body fat or simply fat, is the loose connective tissue composed mostly of adipocytes. Adipose tissue is derived from pre-adipocytes, and is known to cushion and insulate the body. The tissue can be removed by inserting a cannula through the skin and connecting it to a vacuum pump to suction out fatty tissue. The procedure is commonly used in body sculping by removing unwanted deposits of excess fat to improve body appearance, and to smooth irregular or distorted body shapes. Liposuction may also be useful for contouring almost any area of the body, including under the chin, neck, cheeks, upper arms, breasts, abdomen, buttocks, hips, thighs, knees, calves, and ankle areas.

The harvested fat may be reintroduced back into the patient in a process known as adipose tissue transplantation. Using the patient's own tissues reduces or eliminates the need for testing for allergic reactions, and the filling replacement tissue may be permanent. Fat cannot be donated from another person since a recipient's body would reject the donated fat as foreign. A disadvantage of fat injection is that it reabsorbs at an unpredictable degree. Given the decline and drawbacks in the use of foreign substances, such as synthetic materials like silicone and Teflon, as well as the use of foreign tissues, such as bovine collagen, and the advantages of autologous adipose tissue, the interest in and demand for this autologous adipose tissue transplantation continues to increase.

Autologous adipose tissue transplantation is performed by surgeons for various cosmetic and reconstructive procedures. For instance, butt augmentation with fat injections is a current technique. Also called the Brazilian Butt Lift, the procedure involves removing fat from a patient's body and using it to increase the shape of the buttocks. Autologous fat is generally removed from the hips, flanks, abdomen, or lower back. The fat is then purified and injected into the buttocks. Typically, two to three times the fat needed is injected to obtain the desired result. However, the conventional syringe employed for injection of fat is difficult to use in certain positions, and the volume of material inserted is difficult to gauge.

What is needed in the industry is a tissue transplantation device that is ergonomic in design, allowing use of an individual's thumb for advancement of fluid, and providing a mechanism to dispense a predetermine volume of material with each thumb operation.

### SUMMARY

A tissue transplantation device formed from a cylindrical vessel housing a conventional 60 cc disposable syringe with piston. A handle is pivotally attached to the vessel, with a proximal end secured to a puller positioned over steps attached to the piston, and a distal end providing a thumb operated lever for advancing the piston a predetermined distance upon depressing the handle to move the piston. The handle includes a link arm of a length sized to move the puller upon engaging a step a predetermined distance. A spring is coupled to the link arm to maintain the distal end of the handle in a normally raised position. In a preferred embodiment, the link arm and steps are constructed and arranged to advance the piston to cause dispensing of 10 cc of tissue for each handle depression. In this embodiment, a 60 cc syringe is dispensed with six pumps. A cannula is attached to a dispensing end of the syringe for ease of transplantation. A fill port adapter can be positioned at the dispenser end of the cannula; the fill port adapter allowing material to be drawn into the syringe without removal of the cannula.

It is an objective of the invention to provide a tissue transplantation device having an ergonomic handle specifically designed for thumb actuation. In a related feature of the present invention, the syringe includes a ratchet actuator to advance a predetermined amount of fluid with each handle movement.

Yet another objective of the invention is to advance 10 cc of fluid for each pump action, wherein a 60 cc syringe is dispensed with six pumps.

Still another objective of the invention is to provide a tissue transplantation device that can be used with one hand having a handle that provides a balance point.

Still another objective of the invention is to disclose a tissue transplantation device constructed from 6061 aluminum to allow ease of autoclaving.

Another feature of the present invention may be found in a fill port positioned between the syringe and cannula.

Other objectives and advantages of this invention will become apparent from the following description taken in conjunction with any accompanying drawings wherein are set forth, by way of illustration and example, certain embodiments of this invention. Any drawings contained herein constitute a part of this specification, include exemplary embodiments of the present invention, and illustrate various objects and features thereof.

### BRIEF DESCRIPTION OF THE DRAWINGS

FIG. 1 is a perspective view of the adipose tissue transplantation device;

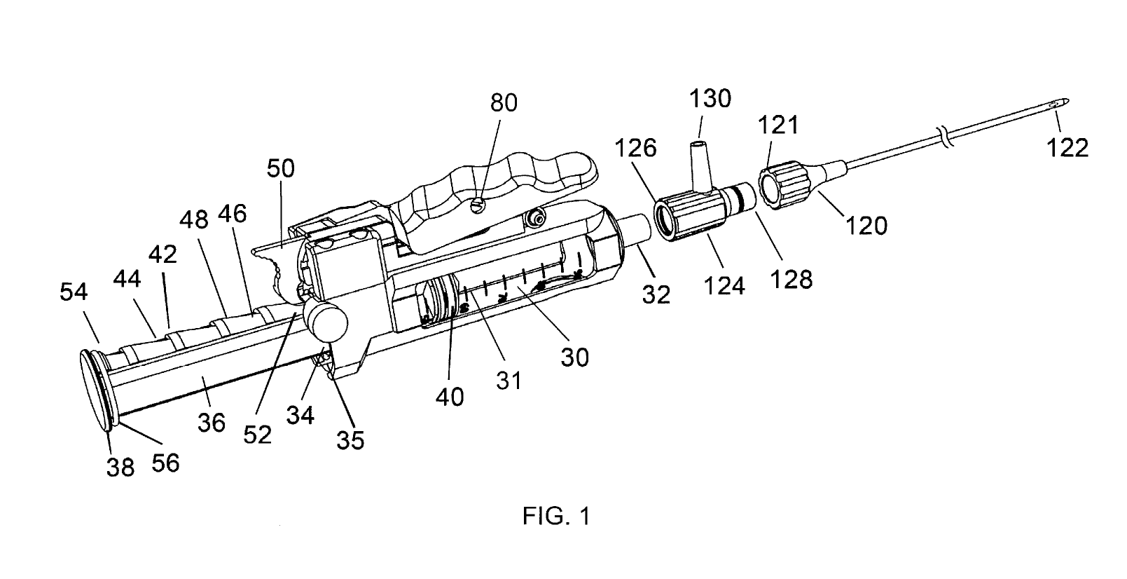

FIG. 2 is a perspective view of the device without a syringe; and

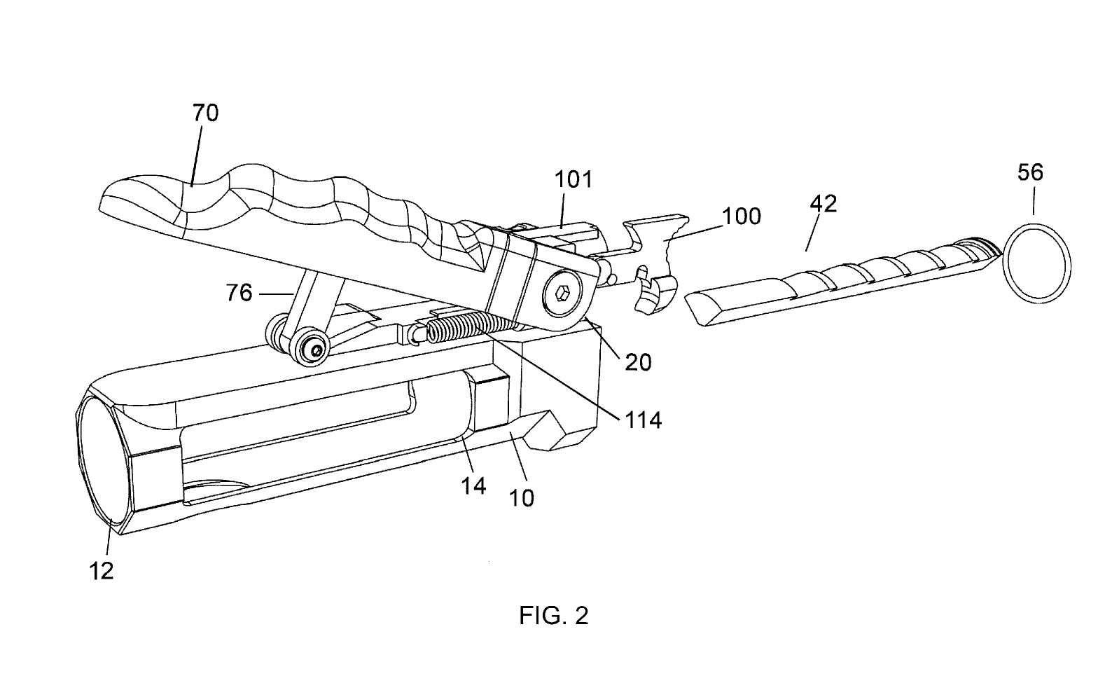

FIG. 3 is an exploded view of FIG. 2.

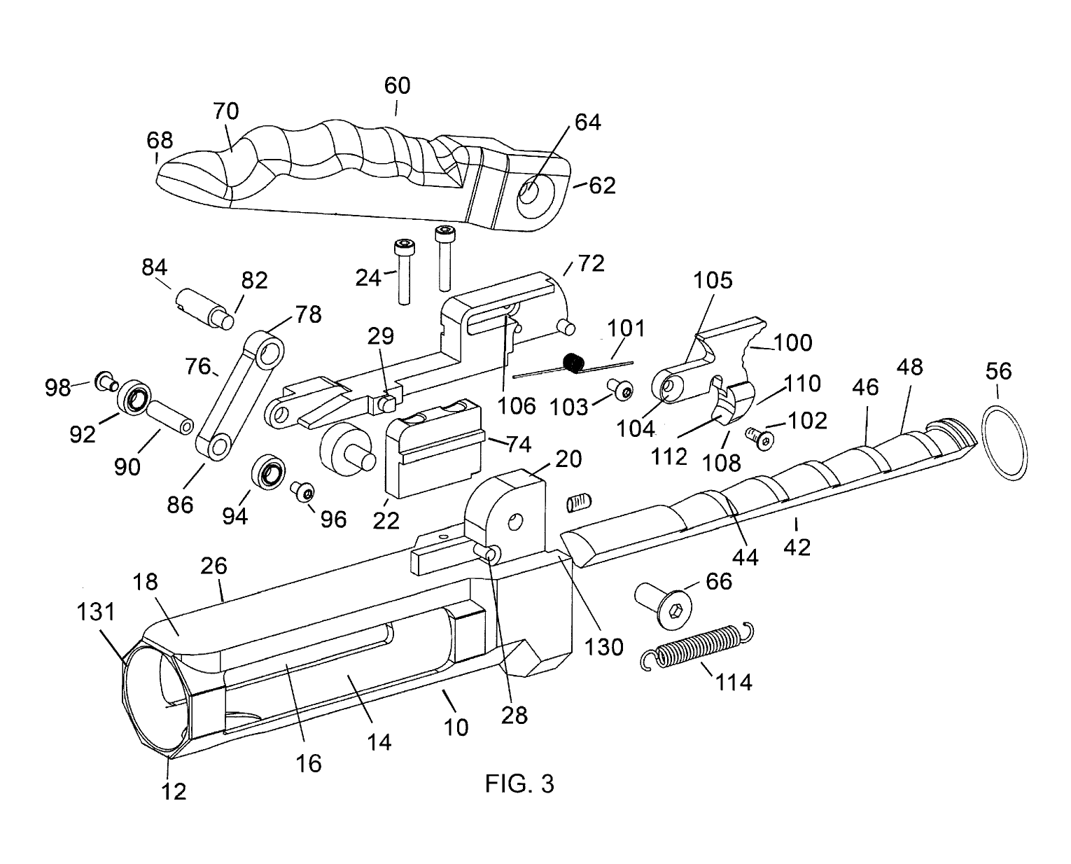

### DETAILED DESCRIPTION OF THE PREFERRED EMBODIMENT

Detailed embodiments of the instant invention are disclosed herein; however, it is to be understood that the disclosed embodiments are merely exemplary of the invention, which may be embodied in various forms. Therefore, specific functional and structural details disclosed herein are not to be interpreted as limiting, but merely as a basis for the claims and as a representation basis for teaching one skilled in the art to variously employ the present invention in virtually any appropriately detailed structure.

Now referring to the Figures, set forth is a cylindrical vessel 10 having a continuous side wall 12 with a first view port 14 formed along a side surface and a second view port 16 formed along a second side. An upper surface 18 includes a first base member 20 formed along an upper portion of the cylindrical vessel 10 and an attachable base 22, which is secured to the first base member 20 by use of a base screw 24. The upper surface 18 further includes a bearing surface 26 and a first spring hook 28. A plastic disposable syringe 30 is positioned within the cylindrical vessel 10, having a dispensing end 32 and an open end 34. The open end 34 receives a piston shaft 36, which is preferably cross shaped having an end 38 commonly used for pushing the piston into the syringe 30, with an opposite end 40 having seals to engage the inner side wall of the syringe 30 for displacing of material placed in the syringe through the dispensing end 32.

The cross shaped piston shaft 36 receives a step member 42, having individual steps 44 formed along the length of the step member 42. Each step has a flat surface 46 and a curved side surface 48 for use in engaging a hook member 50 in a manner to be further explained in this specification. The step member 42 is releasably attached to the cross shaped piston 36, having a first end 52 of the step member insertable within the syringe housing 30, and a second end 54 attached to the cross shaped piston 36 by the use of an O-ring 56. The O-ring 56 essentially operates as a rubber band, hence is used for fastening the second end 54 to the cross shaped piston 36.

A handle 60 has a proximal end 62 with a mounting aperture 64 for attachment to the base members 20, 22 with a cap screw 66. A distal end 68 of the handle 60 is ergonomically shaped for receipt of an individual's thumb by use of a thumb cavity 70, wherein a surgeon would grasp the cylindrical vessel 10 with forefingers and place his/her thumb onto the thumb cavity 70, so as to use the device in an underhand position.

The handle 60 is further attached to the slide member 72, which is positioned beneath a shelf 74 of the second, attachable base member 22. Slide member 72 is positioned along the upper surface 18 of the cylindrical vessel 10, having a link member 76 with an upper end 78 securable to an aperture 80 on the handle 60 by use of an upper link pin 82 and needle bearing 84. A lower end 86 of the link member 76 is attached to the slide member 72 with a sleeve 90 supporting a first roller bearing 92 and second roller bearing 94 secured to the slide member 72 by lower link member screw pins 96 and 98. The slide member 72 includes a second spring hook 29.

A puller member 100 is attached to the slide member 72 with a pulley pin 102 inserted through an aperture 104 on the pulley member 100 for securement to slide member pulley section 106. Puller member 100 includes a step engaging member 108, having a curved front surface 110 and a hook shaped rear surface 112. The hook shaped rear surface 112 engages the step member 42 by movement of the handle, causing the slide member 72 to advance over a step 44 to engage a flat surface 46. Upon release of the handle, the spring member 114 secured to spring hook 28 and 29 pulls the slide member 72 to allow the curved edge surface 110 to slide over the curved side surface 48 and engage a flat surface 46 so as to allow re-engagement of the piston 36. View ports 14 and 16 allow for viewing the contents of the syringe 30, which has fluid marking indicia 31 along the length of the syringe 30. The puller member 100 operates in conjunction with spring member 101 attached to the slide member 72 by attachment screw 103. The spring member 101 biases an upper surface 105 of the puller member 100, so as to engage the curvature of the step member 42 as the step engaging member 108 is slid along the steps of the step member 42.

The step member 42 and link member 76 are constructed and arranged such that depression of the handle will advance the piston to expel 10 cc of fluid from the syringe. In operation, for each depression of the handle 60, the link member 76 engages the roller bearings 92, 94 to roll across bearing surface 26, pulling the slide member 72 by engaging the puller member 100. The hook 108 engages the flat surface 46 of the step member 42, advancing the piston 36 a distance to cause about a 10 cc displacement. In a preferred embodiment, the syringe 30 forms a 60 cc receptacle. The dispensing end 32 is coupled to a cannula 120 for dispensing of fat tissue through apertures 122 located along the length of the cannula 120.

To allow ease of filling, a fill port adapter 124 can be placed between the dispensing end 32 and the attachment end 121 of the cannula 120; the fill port 124 having a first end 126 frictionally engaging the dispensing end 32, and a delivery end 128 frictionally engaging the attachment end 121 of the cannula 120. A tissue receptacle, not shown, can be attached to insert port 130, and upon pulling of piston end 38, fluid is drawn into the syringe body 30. The insert port 130 is then capped, and the handle can be re-engaged to allow dispensing of fluid from the syringe. In the preferred embodiment, the cylindrical vessel 10, slide member 72 and handle are constructed from 6061 aluminum, and the spring is constructed from stainless steel, wherein the device can be autoclaved.

The cylindrical vessel 10 includes a flange 131 for holding the open end 34 lip 35 of the syringe 30. The lip 35 on a conventional syringe is oblong, allowing attachment by rotation of the lip 35 to engage the flange 131.

It is to be understood that while a certain form of the invention is illustrated, it is not to be limited to the specific form or arrangement herein described and shown. It will be apparent to those skilled in the art that various changes may be made without departing from the scope of the invention, and that the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments described herein are presently representative of the preferred embodiments, are intended to be exemplary, and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

The term “coupled” is defined as connected, although not necessarily directly, and not necessarily mechanically. The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

What is claimed is:

1. A tissue transplantation device comprising:
a cylindrical vessel;
a syringe positionable within said vessel, said syringe consisting of a body having a dispensing end and an open end forming a receptacle therebetween, said open end for receipt of a piston constructed and arranged to engage an inner side wall of said body, said piston including a step member positioned along a side edge of said piston;
a handle pivotally attached to said vessel, said handle having a proximal end secured to a puller positioned over said step member, and a distal end having a thumb operated lever for advancing said piston a predetermined distance upon depressing said handle to engage a step of said step member;
wherein fat tissue placed within said receptacle is forced through said dispensing end by movement of said handle for advancement of said piston.

2. The tissue transplantation device according to claim 1, wherein said body is further defined as a disposable 60 cc syringe having a transparent plastic housing with fluid marking indicia.

3. The tissue transplantation device according to claim 1, wherein said handle includes a link arm of a length sized to move said puller upon engaging a step a predetermined distance to displace about 10 cc of fluid volume from said body.

4. The tissue transplantation device according to claim 1, wherein said link arm is coupled to a spring, wherein said link arm maintains said distal end of said handle in a normally raised position.

5. The tissue transplantation device according to claim 1, wherein said puller has a curved frontal surface to allow movement over said steps.

6. The tissue transplantation device according to claim 1, wherein said puller has a hook shaped surface to engage a step.

7. The tissue transplantation device according to claim 1, wherein said distal end includes an ergonomic thumb receptacle.

8. The tissue transplantation device according to claim 1, wherein said steps are formed on an insert securable to said side edge of said piston.

9. The tissue transplantation device according to claim 1, wherein said steps are formed integral with said piston.

10. The tissue transplantation device according to claim 1, wherein said cylindrical vessel includes at least one view port aperture to allow viewing of material placed within the receptacle.

11. The tissue transplantation device according to claim 1, wherein a cannula is attached to said dispensing end.

12. The tissue transplantation device according to claim 11 including a fill port adapter positioned between said dispenser end and said cannula, said fill port adapter allowing material to be drawn into the receptacle without removal of cannula.

13. The tissue transplantation device according to claim 1 wherein said device is constructed from 6061 aluminum.

14. The tissue transplantation device according to claim 1 wherein said proximal end of said handle is pivotally attached to one end of said cylindrical body, and said distal end of said handle is positioned over the opposite end of said cylindrical body.
